(19) United States
(12) Patent Application Publication
(10) Pub. No.: US 2016/0310063 A1
(43) Pub. Date: Oct. 27, 2016

(54) DEVICE AND METHOD FOR DETECTING MONOSODIUM URATE DEPOSITIONS

(71) Applicants: Declan Joseph CURRAN, Limerick (IE); Mark Robert TOWLER, Toronto (CA)

(72) Inventors: Declan Joseph CURRAN, Limerick (IE); Mark Robert TOWLER, Toronto (CA)

(21) Appl. No.: 15/106,224

(22) PCT Filed: Dec. 19, 2014
(86) PCT No.: PCT/CA2014/000906

§ 371 Date: Jun. 17, 2016

Related U.S. Application Data
(60) Provisional application No. 61/918,054, filed on Dec. 19, 2013.

(51) Int. Cl. A61B 5/00

(52) U.S. Cl. CPC A61B 5/4538; A61B 5/0059; A61B 5/0075; A61B 5/4869

(57) ABSTRACT

Various embodiments are described herein for a method and a use of a device for detecting gout. The method can include irradiating a portion of the anatomical region through a skin surface using a light source and receiving scattered light from the portion of the anatomical region. The portion of the anatomical region may be subcutaneous. The method further includes determining Raman spectral data from the received scattered light and identifying a plurality of peaks associated with monosodium urate in the Raman spectral data. The method further comprises determining that a monosodium urate deposition is present in the anatomical region when the number of identified peaks associated with monosodium urate is greater than a detection threshold. The method may be performed using a device that includes a light source, an optical detection component and a spectral selection component.

### FIELD

The various embodiments described herein generally relate to methods for detecting monosodium urate depositions and uses of a device for detecting monosodium urate depositions, which may be indicative of various medical conditions such as, but not limited to, gout, for example.

### BACKGROUND

Uric Acid (UA) is a metabolite of purine, which is found in high concentrations in meat and meat products. Certain diets can result in the increased intake of purines that can lead to negative effects when a person's body is unable to get rid of purine by-products. When overproduction or under-excretion of uric acid occurs, the serum urate (SU) concentration may exceed the solubility of urate (a concentration of approximately >6.8 mg/dL). As a result a supersaturation state can occur; which is called hyperuricemia. Hyperuricemia can lead to deposits of urate salts, such as monosodium urate (MSU) in a person's joints resulting in gout. In addition, hyperuricemia can occur in conjunction with, or be a precursor to, obesity, diabetes mellitus, and hypertonia, and carries an increased risk of cardiovascular problems. Hence, early prediction of hyperuricemia is imperative to maintain physical health.

Gout is an inflammatory disease triggered by deposits of MSU crystals that are caused by elevated levels of uric acid in the blood (hyperuricemia). Gout can proliferate undetected until a critical level of MSU crystal build-up is reached, after which a patient can suffer from attacks of gout. These attacks can progress to chronic gout and, if left untreated, may potentially lead to further health problems such as joint damage and comorbidities including atherosclerosis, hyperlipidemia, hypertension, obesity and organ failure. However, if sufferers are diagnosed early and properly treated the prognosis is generally positive.

The primary diagnostic test for gout employed by medical professionals requires synovial fluid (SF) to be extracted from an affected joint and analyzed under a microscope for the presence of MSU crystals. This is a painful and invasive procedure that requires inserting a needle into a person's joint to extract the SF. This technique has also been shown to have poor accuracy. It is quite common for MSU levels to be normal or low during an attack, so the best time to perform this diagnostic test is 2 to 3 weeks after an attack. Accordingly, there is also an added element of timing for this technique to be successful.

As a result of the difficulties associated with current diagnostic tests, medical professionals often diagnose gout based on clinical features of the phalangeal joints and hyperuricemia. Typically this involves following the Rome, New York or EULAR criteria. Unfortunately, these criteria have been shown to have poor accuracy and both require gout and hyperuricemia to be in the advanced stages to be accurate. Accordingly, crystal identification in extracted SF is still considered the gold standard for diagnosing gout, despite its invasive nature and poor reliability.

### SUMMARY OF VARIOUS EMBODIMENTS

In a broad aspect, at least one embodiment described herein provides a method of detecting monosodium urate depositions in an anatomical region. The method comprises irradiating a portion of the anatomical region through a skin surface using a light source and receiving scattered light from the portion of the anatomical region. The portion of the anatomical region may be subcutaneous. The method further includes determining Raman spectral data from the received scattered light and identifying a plurality of peaks associated with monosodium urate in the Raman spectral data. The method further comprises determining that a monosodium urate deposition is present in the anatomical region when the number of identified peaks associated with monosodium urate is greater than a detection threshold.

In some cases, the plurality of peaks associated with monosodium urate includes at least one of peaks at 1502 cm⁻¹, 1445 cm⁻¹, 1420 cm⁻¹, 1205 cm⁻¹, 1060 cm⁻¹, and 1010 cm⁻¹.

In some cases, the detection threshold can be at least 6 peaks. In some cases, the monosodium urate deposition can be determined to be present in the anatomical region when the identified peaks include each of the peaks at 1502 cm⁻¹, 1445 cm⁻¹, 1420 cm⁻¹, 1205 cm⁻¹, 1060 cm⁻¹, and 1010 cm⁻¹.

In some cases, the acts of irradiating, receiving scattered light, and determining Raman spectral data can be performed at least twice. In such cases, the method can include averaging the determined Raman spectral data and identifying the plurality of peaks associated with monosodium urate from the averaged Raman spectral data.

In some cases, the method can also include displaying a Raman spectrum based on the determined Raman spectral data and identifying the plurality of peaks associated with monosodium urate from the displayed Raman spectrum.

In some cases, the Raman spectral data can be filtered to exclude data outside of at least one band-limited region of interest. The plurality of peaks associated with monosodium urate can be identified in the Raman spectral data from the filtered at least one band-limited region of interest. In some cases, the at least one band-limited region of interest can include at least one of a first region of interest from 500 cm⁻¹ to 700 cm⁻¹ and a second region of interest from 1000 cm⁻¹ to 1502 cm⁻¹.

In some cases, the method comprises irradiating an anatomical region using a light source, receiving scattered light from the anatomical region, determining Raman spectral data associated with the received light that includes at least one of a first region of interest from 500 cm-1 to 700 cm-1 and a second region of interest from 1000 cm-1 to 1502 cm-1, identifying a plurality of peaks in the at least one of the first region of interest and the second region of interest and determining that a monosodium urate deposition is present in the anatomical region when the plurality of peaks includes at least one peak associated with monosodium urate. In some cases, the Raman spectral data includes both the first region of interest from 500 cm⁻¹ to 700 cm⁻¹ and the second region of interest from 1000 cm⁻¹ to 1502 cm⁻¹. In some cases, the Raman spectral data is filtered to exclude data outside of the at least one of the first region of interest and the second region of interest and the peaks are identified from the filtered Raman spectral data.

In some cases, the method further includes determining that a monosodium urate deposition is present in the anatomical region when the identified plurality of peaks includes at least one of peaks at 1502 cm⁻¹, 1445 cm⁻¹, 1420 cm⁻¹, 1205 cm⁻¹, 1060 cm⁻¹, and 1010 cm⁻¹. In some cases, the monosodium urate deposition is determined to be present when the identified plurality of peaks includes each of peaks at 1502 cm⁻¹, 1445 cm⁻¹, 1420 cm⁻¹, 1205 cm⁻¹, 1060 cm⁻¹, and 1010 cm⁻¹.

In another broad aspect, at least one embodiment described herein provides a use of a device to perform a method of detecting monosodium urate depositions in an anatomical region, wherein the method is described herein. The device comprises a light source configured to irradiate an anatomical region and an optical detection component configured to receive scattered light from the anatomical region. The device further comprises a spectral selection component configured to determine at least one peak of interest in Raman spectral data associated with the scattered light.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now briefly described.

FIG. 1 is a block diagram of an example embodiment of a system that can detect monosodium urate depositions non-invasively.

FIG. 2 is a flowchart of an example embodiment of a method for detecting monosodium urate depositions.

FIG. 3A is a diagram illustrating an example plot of a Raman spectrum of uric acid.

FIG. 3B is a diagram illustrating an example plot of a Raman spectrum of monosodium urate (MSU).

FIG. 4A is a diagram illustrating an example plot of Raman spectrums of ayes flesh and bone through ayes flesh.

FIG. 4B is a diagram illustrating an example plot of a Raman spectrum of uric acid salts.

FIG. 4C is a diagram illustrating an example plot of a Raman spectrum of uric acid through ayes flesh.

FIG. 5 is a diagram illustrating an example plot of a Raman spectrum of tophus fluid extracted from a patient suffering from gout.

FIG. 6 is a diagram illustrating an example plot of a Raman spectrum acquired from the metatarsophalangeal joint of a clinically diagnosed gout patient.

FIG. 7 is a diagram illustrating an example plot of a Raman spectrum of tophus fluid extracted from a patient suffering from gout with regions of interest identified.

FIG. 8 is a diagram illustrating an example plot of a Raman spectrum of a tophi deposit of a patient suffering from gout.

Further aspects and features of the embodiments described herein will appear from the following description taken together with the accompanying drawings.

### DETAILED DESCRIPTION OF THE EMBODIMENTS

Various apparatuses or methods will be described below to provide an example of an embodiment of the claimed subject matter. No embodiment described below limits any claimed subject matter and any claimed subject matter may cover methods or apparatuses that differ from those described below. The claimed subject matter is not limited to apparatuses or methods having all of the features of any one apparatus or methods described below or to features common to multiple or all of the apparatuses or methods described below. It is possible that an apparatus or methods described below is not an embodiment that is recited in any claimed subject matter. Any subject matter disclosed in an apparatus or method described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices can be directly connected to one another or connected to one another through one or more intermediate elements or devices via an electrical element, electrical signal or a mechanical element depending on the particular context. Furthermore, the term “communicative coupling” indicates that an element or device can electrically, optically, or wirelessly send data to another element or device as well as receive data from another element or device.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Described herein are various example embodiments of a system and method that can be used for detecting monosodium urate depositions in an anatomical region of an individual such as a patient. Depositions of monosodium urate crystals below the skin surface are indicative of gout and hyperuricemia (the underlying condition that produces gout) as well as increasing the possibility of other conditions such as obesity, diabetes mellitus, hypertonia, and pre-eclampsia. The systems and methods described herein can be used to detect monosodium urate depositions, and thereby gout, in an efficient and non-invasive manner. These systems and methods can allow conditions such as gout to be diagnosed in patients quickly and easily. Furthermore, such methods may allow for a much broader spectrum of medical professionals to accurately diagnose gout without having to rely on clinical features of gout presenting in patients.

Generally, the systems and methods described herein are configured to use Raman Spectroscopy (RS) to determine the presence of MSU depositions in an anatomical region of a patient. RS is a technique whereby incident light, made up of photons, can be absorbed or scattered by a material when the energy of an incident photon corresponds to the energy gap between the ground state and excited state of a molecule in the material being irradiated. As used herein, the term light refers to any portion of the electromagnetic spectrum in the infrared, visible or ultraviolet regions.

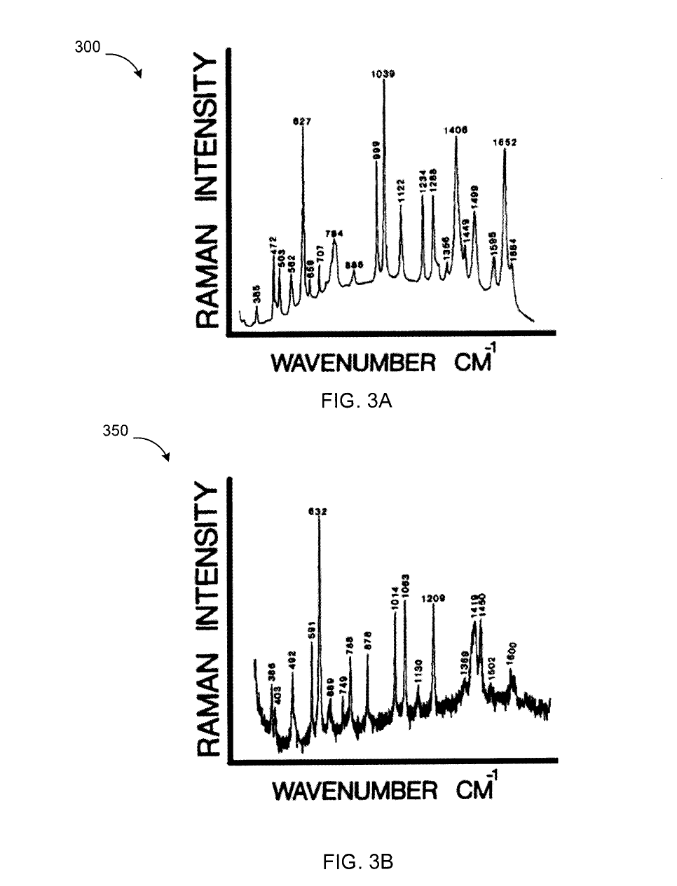

A single frequency of radiation (i.e. monochromatic light) can be used to irradiate the anatomical region being investigated. The anatomical region will scatter the incident light and a small portion of the scattered light will be shifted in energy with respect to the source beam. Scattering can take the form of Rayleigh scattering or Raman scattering. The Raman scattered light can be separated from the Rayleigh scattered light using a wide variety of optical components known in the art, such as prisms, optical gratings, filters etc. Plotting the Raman scattered light against the frequency shift results in a Raman spectrum which can be considered a “fingerprint” of the material. For example, as shown below in FIG. 3, a Raman spectrum can be used to determine and differentiate uric acid (UA) and MSU.

Identifying materials in sub-cutaneous anatomical regions, such as joints, renders the task more complex. The depth of a particular anatomical region with regard to the surface of the skin is increased and can vary from person to person. The presence of other materials such as cartilage, synovial fluid and bone may also obscure the information of interest in the Raman spectral data that is collected when examining the anatomical region.

Furthermore, a difficult incident angle of radiation may exist depending on the anatomical region being examined. For example, where a laser focuses at approximately 2.8 cm away from the porthole on a Raman spectral device, one may position the device so that it is about 2.8 cm away from the anatomical region under evaluation.

In some cases, the systems and methods described herein can use a Raman spectroscopy device such as a Raman laser device or offset Raman laser device. This can ensure that the signal recorded is that of the surface of the knuckle by moving the device or the patient relative to one another to ensure the Raman device focuses on the appropriate anatomical region. This is the region where MSU crystals are expected to be most prevalent, as opposed to the skin, cartilage or bone. In some cases, low levels of MSU build-up may produce a relatively weak signal in the scattered light (and thus be present as a weak signal in the Raman spectral data determined from the scattered light). Accordingly, in accordance with the teachings herein, signal preprocessing, which may include digital signal analysis, may be used to filter out the characteristic Raman traces of the natural tissues of the articular joint such as, but not limited to, skin, cartilage, blood, and synovial fluid, for example, to highlight the Raman spectral data associated with any MSU deposits that may be present at the anatomical region.

The systems and methods described herein enable the substantially instantaneous detection of MSU deposits. This allows users to diagnose gout and its underlying cause, hyperuricemia, as well as possibly other medical conditions. These systems and methods can be used by primary care physician, or other medical professionals, without significant training to identify depositions of MSU crystals at affected joints in a rapid, non-invasive manner with no side effects.

The systems and methods described herein for the determination of MSU crystals can be done much faster than the gold standard which requires the synovial fluid to be extracted and then processed. Typically, the gold standard method involves centrifugation of extracted synovial fluid to compact any crystals. The centrifuged fluid is then placed on a microscope slide and evaluated using polarized light microscopy. Polarized light microscopy generally entails searching through the centrifuged fluid to identify thin, acicular crystals which are birefringent under polarized light. This method is costly, time consuming and often requires specific training on the part of the medical professional or analysis by an offsite technician.

In accordance with the teachings herein, non-invasive Raman spectroscopy can be used to quickly identify the presence of MSU crystals for diagnosis of gout or hyperuricemia or in prognostic assays prior to, during, or after disease therapy or lifestyle alterations to minimize or reverse disease progression. By directing a Raman light source (such as a Raman laser) at the skin directly above the anatomical region in question (e.g. a joint around which MSU crystals may be agglomerated) and focusing the light source on the sub-cutaneous region being investigated, hyperuricemia and gout may be identified at a much earlier stage of development. MSU depositions can be identified without requiring individual crystals to break away from the deposit on the joint and float into the SF.

This also allows MSU crystals to be identified in many different regions of a patient's body, either in synovial fluid or as tophaceous gout deposits. While rheumatologists may be trained to aspirate fluid from body joints, point of care providers such as emergency room doctors and general practitioners are typically not trained to aspirate joints that are smaller than a knee. This is particularly problematic for the detection of MSU deposits, which are most commonly found in toe or finger joints. However, this challenge is addressed by the teachings herein as aspiration is not required.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile and non-volatile memory and/or storage elements). These devices may also have at least one input device (e.g. a keyboard, a mouse, a touchscreen, and the like), and at least one output device (e.g. a display screen, a printer, a wireless radio, and the like) depending on the nature of the device.

It should also be noted that there may be some elements that are used to implement at least part of one of the embodiments described herein that may be implemented via software that is written in a high-level procedural language such as object oriented programming. Accordingly, the program code may be written in C, C⁺⁺ or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

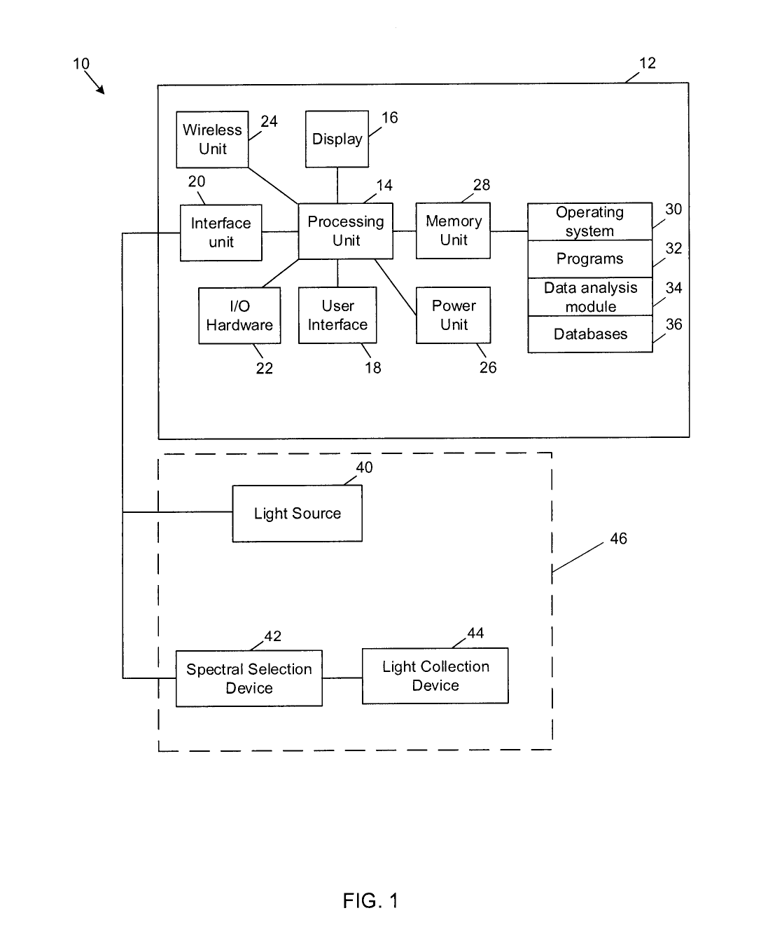

Referring now to FIG. 1, shown therein is a block diagram of an example embodiment of a detection system 10 (or in some embodiments a detection device 10) that can be used to determine whether MSU deposits are present in an anatomical region of a patient. The system 10 includes an operator unit 12, a light source 40, a light collection device 44 and a spectral selection device 42. The operator unit 12, light source 40, light collection device 44 and spectral selection device 42 can be provided together as a device 10 that can be used to detect MSU deposits and thereby enable medical professionals to diagnose conditions such as gout or hyperuricemia. In some embodiments, the operator unit 12, light source 40, light collection device 44 and spectral selection device 42 may be provided as a combined diagnostic device, while in other embodiments one or more of the operator unit 12, light source 40, light collection device 44 and Spectral selection device 42 may be provided as separate units coupled together in system 10.

The system 10 is provided as an example and there can be other embodiments of the system 10 with different components or a different configuration of the components described herein. The system 10 further includes several power supplies (not all shown) connected to various components of the system 10 for providing power thereto as is commonly known to those skilled in the art. In general, a user, such as a medical professional, may interact with the operator unit 12 to perform various acts of a method for detecting MSU deposits for an anatomical region of interest. For example, the user may interact with the operator unit 12 to align the device 10 with an anatomical region of a patient, activate the light source 40 to irradiate the anatomical region, receive scattered light from the anatomical region using the light collection device 40, determine Raman spectral data from the scattered light, and identify a plurality of peaks in the Raman spectral data. The presence of MSU deposits in the anatomical region may be determined if the identified peaks include a number of peaks associated with MSU greater than a detection threshold.

In various embodiments, the operator unit 12 comprises a processing unit 14, a display 16, a user interface 18, an interface unit 20, Input/Output (I/O) hardware 22, a wireless unit 24, a power unit 26 and a memory unit 28. The memory unit 28 comprises software code for implementing an operating system 30, various programs 32, a data analysis module 34, and one or more databases 36. Many components of the operator unit 12 can be implemented using a desktop computer, a laptop, a mobile device, a tablet, and the like.

The processing unit 14 controls the operation of the operator unit 12 and can be any suitable processor, controller or digital signal processor that can provide sufficient processing power processor depending on the configuration, purposes and requirements of the system 10 as is known by those skilled in the art. For example, the processing unit 14 may be a high performance general processor. In alternative embodiments, the processing unit 14 may include more than one processor with each processor being configured to perform different dedicated tasks. In alternative embodiments, specialized hardware can be used to provide some of the functions provided by the processing unit 14.

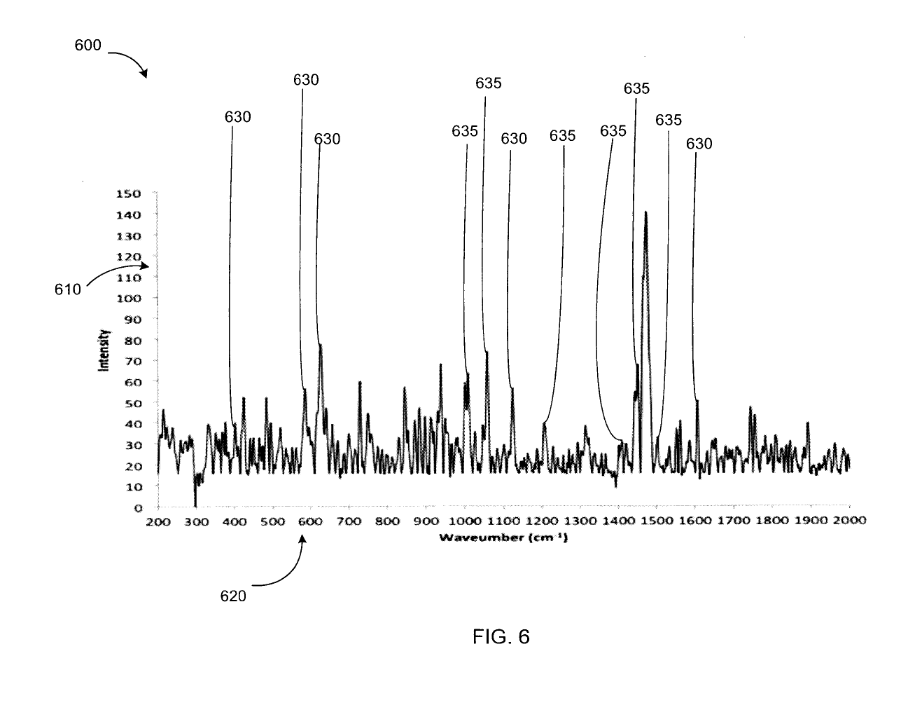

The display 16 may be any suitable display that provides visual information depending on the configuration of the operator unit 12. For instance, the display 16 may be a cathode ray tube, a flat-screen monitor and the like if the operator unit 12 is a desktop computer. In other cases, the display 16 may be a display suitable for a laptop, tablet or handheld device such as an LCD-based display and the like. In some embodiments, the display 16 may be used to display a Raman spectrum determined based on Raman spectral data collected from an anatomical region, such as the example Raman spectrum plot shown in FIG. 6.

The user interface 18 may include at least one of a mouse, a keyboard, a touch screen, a thumbwheel, a track-pad, a track-ball, a card-reader, voice recognition software and the like again depending on the particular implementation of the operator unit 12. In some cases, some of these components can be integrated with one another.

The interface unit 20 may be any interface that allows the operator unit 12 to communicate with other devices or computers. In some cases, the interface unit 20 may include at least one of a serial port, a parallel port or a USB port that provides USB connectivity. The interface unit 20 may also include at least one of an Internet, Local Area Network (LAN), Ethernet, Firewire, modem or digital subscriber line connection. Various combinations of these elements may be incorporated within the interface unit 20.

The I/O hardware 22 is optional and can include, but is not limited to, at least one of a microphone, a speaker and a printer, for example. For example, I/O hardware 22 can be used to provide feedback indicating that scattered light has been collected or that Raman spectral data has been determined. In some cases, the I/O hardware 22 could also be used to output the Raman spectral data or a Raman spectrum plot that is determined based on the Raman spectral data.

The wireless unit 24 is optional and can be a radio that communicates utilizing CDMA, GSM, GPRS or Bluetooth protocol according to standards such as IEEE 802.11a, 802.11b, 802.11g, or 802.11n. The wireless unit 24 can be used by the operator unit 12 to communicate with other devices or computers.

The power unit 26 can be any suitable power source that provides power to the operator unit 12 such as a power adaptor or a rechargeable battery pack depending on the implementation of the operator unit 12 as is known by those skilled in the art.

The memory unit 28 can include RAM, ROM, one or more hard drives, one or more flash drives or some other suitable data storage elements such as disk drives, etc. The memory unit 28 may be used to store an operating system 30 and programs 32 as is commonly known by those skilled in the art. For instance, the operating system 30 provides various basic operational processes for the operator unit 12. The programs 32 include various user programs so that a user can interact with the operator unit 12 to perform various functions such as, but not limited to, acquiring data, viewing and manipulating data, adjusting parameters for data analysis as well as sending messages as the case may be. For example, the programs 32 may include programs such as GRAMS/AI™ Spectroscopy Software from Thermo Scientific™ for generating a Raman spectrum from collected data.

The data analysis module 34 is used to determine Raman spectral data associated with the scattered light collected by the light collection device 44. The data analysis module 34 receives data that is obtained by the light collection device 44 (or another similar device) to perform this task. In some cases, the data analysis module 34 can be incorporated into the spectral selection device 42 to determine the Raman spectral data from the scattered light collected. For example, the data analysis module 34 may operate along with optical components of the spectral selection device 42 to separate the Raman spectral data from Rayleigh scattered light included in the scattered light received from the light collection device 44.

The data analysis module 34 generally determines a Raman spectrum based on the Raman spectral data. The Raman spectrum may be displayed to a user of device 10 using display 16. The data analysis module 34 may also perform signal processing to smooth or filter the Raman spectral data or Raman spectrum. For example, the data analysis module 34 may use programs 32 such as the GRAMS/AI™ Spectroscopy Software or other spectroscopy software to filter the acquired Raman spectral data as desired.

In some cases, the Raman spectral data may be preprocessed by the spectral selection device 42 or the data analysis module 34. The preprocessing that is done may include standard signal processing techniques such as, but not limited to, at least one of amplification, filtering and de-noising (e.g. averaging) using parameters that depend on the particular signals that are acquired. For example, the preprocessing may include standard signal processing software for removing false signal peaks and identifying and removing contributions to the signal peaks from signals internal to the device 10.

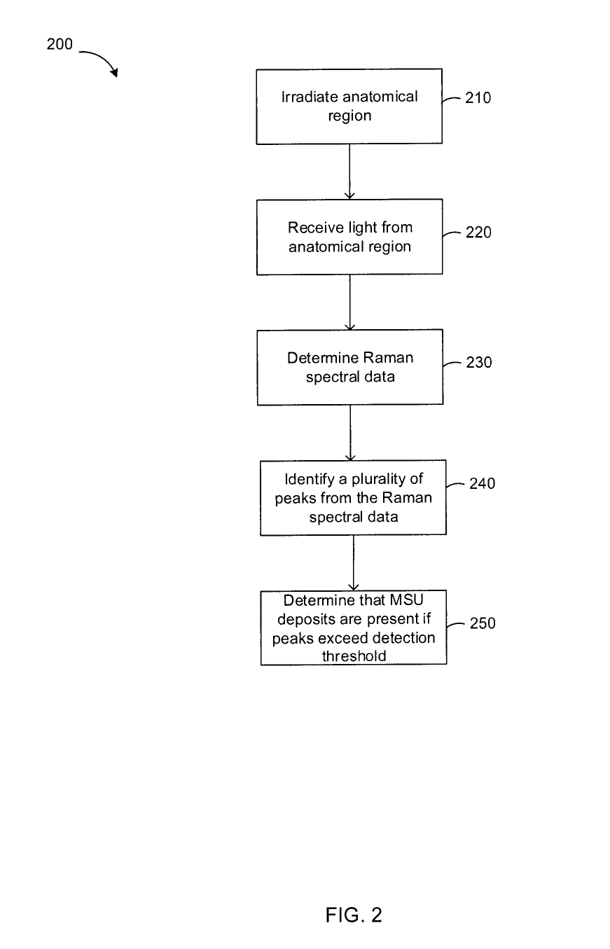

In some cases, Raman spectral data may be acquired in multiple sets. For example, the light source 40 may be configured to irradiate the anatomical region being investigated for a first period of time, the light collection device 44 can collect scattered light during this first time period, and the spectral selection device 42 can determine the Raman spectral data for this first time period. This process can be repeated multiple times, with Raman spectral data determined for each time period. The preprocessing may then include averaging the Raman spectral data from each time period to generate averaged Raman spectral data. In some cases, the Raman spectral data or the averaged Raman spectral data can be used to generate a Raman spectrum. The Raman spectral data can be used to determine if MSU deposits are present in an anatomical region, for example using embodiments of the method described below with reference to FIG. 2.

The analysis module 34 may typically be implemented using software, but there may be instances in which it is implemented using FPGA or application specific circuitry. For ease of understanding, certain aspects of the methods described herein may be described as being performed by the data analysis module 34. It should be noted, however that these methods are not limited in that respect, and the various aspects of the methods described herein may be performed by other modules for detecting MSU deposits, such as the spectral selection device 42.

The databases 36 can be used to store data for the system 10 such as, but not limited to, system settings, parameter values, and calibration data, for example. The databases 36 can also store other information required for the operation of the programs 32 or the operating system 30 such as dynamically linked libraries and the like.

The operator unit 12 comprises at least one interface that the processing unit 14 communicates with in order to receive or send information. This interface can be the user interface 18, the interface unit 20 or the wireless unit 24. For instance, the various parameters used by the system 10 in order to detect MSU deposits in an anatomical region, such as the wavelength of light used to irradiate the anatomical region, may be inputted by a user through the user interface 18 or they may be received through the interface unit 20 from a computing device. The processing unit 14 can communicate with either one of these interfaces as well as the display 16 or the I/O hardware 22 in order to output information related to acquired Raman spectral data, Raman spectrums, and system operating parameters. In addition, users of the operator unit 12 can communicate information across a network connection to a remote system for storage and/or further analysis in some embodiments. This communication may also include email communication.

The user can also use the operator unit 12 to input information needed for system parameters that are needed for proper operation of the system 10 such as calibration information and other system operating parameters as is known by those skilled in the art. Data that are obtained from tests, as well as parameters used for operation of the system 10, may be stored in the memory unit 28. The stored data may include raw recorded data (e.g. scattered light data), preprocessed Raman spectral data as well as processed Raman spectrums.

In some cases, the light source 40, the light collection device 44 and spectral selection device 42 can be provided as a combined Raman unit 46. In some cases, the combined Raman unit 46 may also be combined with the operator unit 12 as a stand-alone detection device 10 that can be used to perform the various methods described herein for detecting MSU deposits. Such a stand-alone detection device 10 may be used by a medical professional to provide a quick and reliable diagnosis of gout, hyperuricemia or other MSU-related condition in a patient. In some cases, the combined Raman unit 46 may be a commercial Raman spectroscopy unit such as a Sierra Series™ spectroscopic reader provided by Snowy Range Instruments®.

The light source 40 comprises hardware and circuitry used to generate light for irradiating the anatomical region of interest. For example, the light source 40 may be a laser source that generates a beam of electromagnetic radiation at a desired wavelength. In some cases, the light source 40 may be tunable such that the desired wavelength can be adjusted, for example using the operator unit 12. In the embodiments using a Sierra Series™ spectroscopic reader, the light source 40 can provide light with a wavelength of 532 nm, 638 nm, 785 nm, 808 nm, or 830 nm.

Generally, any light source 40 capable of generating a strong and relatively monochromatic beam of light can be used in the system 10. Accordingly, the light source 40 is not limited to a laser light source but may also be implemented using other known light generation systems, such as a light projection system, for example.

In laser Raman spectroscopy, monochromatic laser light that is scattered off the surface of an interrogated material is collected by an optical detection system such as the light collection device 44. Most of the light scattered off the material being investigated is scattered elastically at the same wavelength as the initial irradiating light in a process known as Rayleigh scattering. The remainder of the scattered light is scattered inelastically at a different wavelength in a process known as Raman scattering. The Rayleigh scattered light and Raman scattered light can be separated from each other using any suitable wavelength selection system, such as prisms, filters, or optical gratings. The Raman spectral data can thus be obtained from the scattered light collected by the light collection device 44 as data corresponding to the portion of the scattered light that was scattered inelastically by Raman scattering. The Raman spectral data can be used to generate a Raman spectrum that can then be used to identify and quantify concentrations of various substances within the interrogated material.

The light collection device 44 is operable to collect scattered light from the anatomical region of interest. For example, the light collection device 44 may use various optical components, as is known to those skilled in the art, to collect the light scattered by the anatomical region and to direct the scattered light to the spectral selection device 42.

The spectral selection device 42 can be used to separate and select the Raman spectral data from the scattered light collected by the light collection device 44. The spectral selection device 42 may include any number of optical filters used to separate the portion of the scattered light that was Raman scattered, such as optical gratings, filters and prisms, for example. In some cases, the spectral selection device 42 may provide Raman spectral data to the operator unit 12 for further processing or analysis. In some cases, the Raman spectral data can be converted into a Raman spectrum signal that may be displayed visually, for example using display 16. Alternatively, the Raman spectral data can be converted into digital or other numerical formats for further processing or analysis.

Referring now to FIG. 2, shown therein is an example embodiment of a method 200 for detecting MSU deposits in an anatomical region of a patient. The device 10 is an example of a device that can be used to implement the method 200.

At 210, the device 10 can be used to irradiate the anatomical region. In most cases, the device 10 will be used to irradiate a portion of the anatomical region through a skin surface using the light source 40. The portion of the anatomical region that receives the irradiated light may be subcutaneous but in other cases the joints, such as the elbow or a toe, or cartilage such as an MSU buildup in the ear. For example, the light source 40 can be configured to generate a beam of monochromatic light. The wavelength of the light generated can be selected by a user of device 10. In some cases, the monochromatic light generated by the light source 40 will be laser light.

It should be understood that in the various embodiments, different types of light sources with different intensities, frequencies, wavelengths, etc. may be used in some situations as they may work better by causing less fluorescence from the skin. Furthermore, in some cases, the monochromatic light generated by the light source 40 may be laser light.

The light source 40 can be targeted at an anatomical region of interest and the beam of light can be directed to the anatomical region. In some cases, the light source 40 may be adjusted until the correct focus is obtained at a subcutaneous portion of the anatomical region of interest. The light source 40 can be maintained in a fixed position relative to the anatomical region while the anatomical region is being irradiated. This may ensure that the angle of incidence of the light irradiating the anatomical region is consistent. For example, the light source 40 and the light collection device 44 can be held at a consistent distance from the anatomical region being investigated, such as a joint for example. The particular distance from the joint may depend on the particular focal length of the light source 40 being used.

Generally, any anatomical region near to the skin surface can be analyzed by maintaining an MSU detection device in a fixed position relative to the anatomical region for a sufficient period to capture Raman spectral data. For example, this may entail maintaining a device, such as device 10, at a fixed distance from the anatomical region being investigated for 20 s in some cases. Examples of anatomical regions include toes, finger, knee and elbow joints as well as soft tissues such as that found in the elbows, knees, ears and eyebrows. Gout can deposit as tophii, which are nodular masses of monosodium urate crystals in the soft tissues of the body. They are a late complication of hyperuricemia and develop in more than half of patients with untreated gout. These can often appear at elbows, knees, even ears and on the eyebrow.

The light will be scattered by the materials in and around the subcutaneous portion of the region of interest, such as the skin, cartilage, and bone etc. At least some of the scattered light will return to the area of the device 10. The Raman shift generated by a particular material is independent of the wavelength of the light used to irradiate the material. As a result, in general any light source 40 capable of generating a strong and relatively monochromatic beam of light can be used at 210.

At 220, the light collection device 44 can be used to receive and collected the scattered light from the anatomical region, including scattered light from the subcutaneous portion of the anatomical region. As mentioned above, the scattered light will include both Raman scattered light (Raman spectral data) and Rayleigh scattered light. The light collection device 44 may direct the scattered light to the spectral selection device 42 for further processing. The light collection device 44 can also be maintained in a fixed position relative to the anatomical region during the period of time for which the scattered light is being collected.

At 230, Raman spectral data associated with the scattered light collected at 220 is determined from the scattered light. The scattered light can be provided to a spectral selection device 42 that includes various optical components such as prisms, filters and optical gratings. The spectral selection device 42 can then separate the Rayleigh scattered light and the Raman scattered light and provide Raman spectral data. The Raman spectral data can be used to determine whether MSU crystals are present in the anatomical region being investigated, thereby providing a diagnosis of gout, hyperuricemia and other MSU-related conditions.

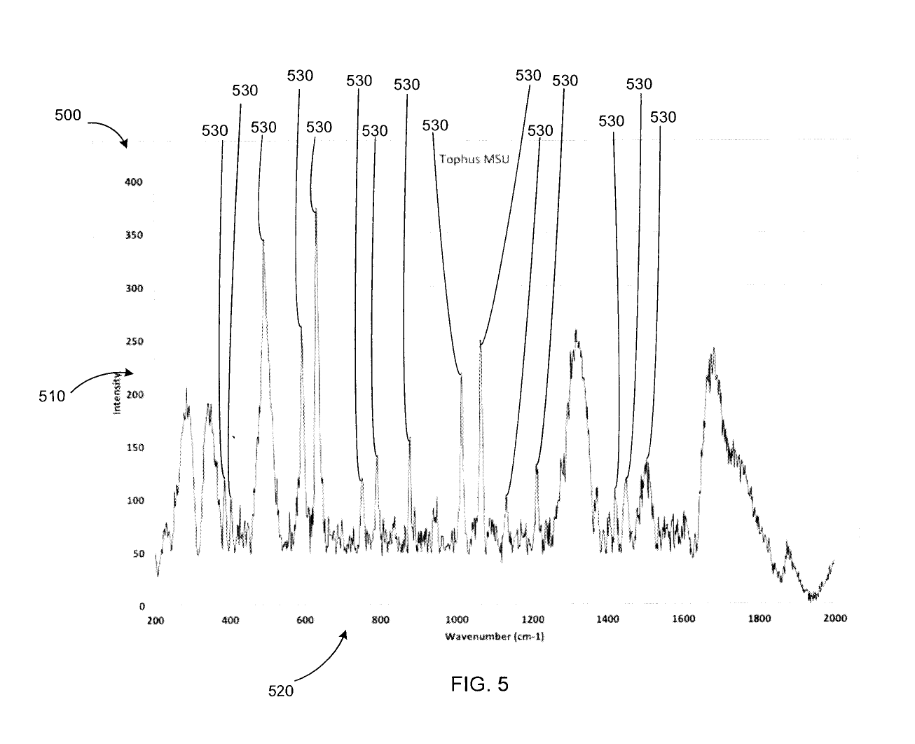

In some cases, the Raman spectral data can be used to generate a Raman spectrum that can be displayed to a user. For example, the Raman spectral data may be used to generate a Raman spectrum such as the Raman spectrum shown in FIGS. 5 and 6 discussed below.

In some cases, the irradiation of the anatomical region, receiving scattered light and determining Raman spectral data can be repeated a number of times for a particular anatomical region. For example, the anatomical region can be irradiated 5 separate times for a period of 10 seconds each, the scattered light can be received and collected for each time period, and the Raman spectral data associated with the scattered light can be determined for each time period. In such cases, the method 200 may further include averaging the determined Raman spectral data for each time period to generate averaged Raman spectral data.

At 240, a plurality of peaks are identified in the Raman spectral data. For example, the plurality of peaks identified may be peaks associated with MSU. As mentioned above, a Raman spectrum can be determined based on the Raman spectral data. The peaks of interest may be identified by using known automated peak identification methods but looking for at least one peak in a certain wavelength region. Alternatively, the peaks may be displayed in a spectrum on a monitor such as display 16 or provided via I/O hardware 22 as a hardcopy and then inspected.

The plurality of peaks that are identified may include one or more peaks associated with MSU crystals or uric acid in vivo. However, the peaks associated with lab grade MSU (see FIG. 3B below) and biologically deposited MSU (see FIG. 6 below) can have slightly different peak locations. In some cases, the peaks may shift slightly by up to 5 cm⁻¹. Accordingly the detection of peaks associated with MSU may include a range of +/−5 cm⁻¹ around each of the peaks. This may account for slight shifts in the Raman spectral data that may be caused by minor differences between tophi deposits as compared with other MSU deposits, where stretching or compression of bonds in the crystals could explain the slight shift in Raman spectral data.

For example, the plurality of peaks associated with MSU may include at least one of peaks at 1502 cm⁻¹, 1445 cm⁻¹, 1420 cm⁻¹, 1205 cm⁻¹, 1060 cm⁻¹, and 1010 cm⁻¹. Accordingly, in such examples detection of the plurality of peaks associated with MSU may include a range of +/−5 cm⁻¹ around each of the peaks at 1502 cm⁻¹, 1445 cm⁻¹, 1420 cm⁻¹, 1205 cm⁻¹, 1060 cm⁻¹, and 1010 cm⁻¹.

In some cases, the Raman spectrum may be displayed to a user of the device 10 using the display 16. In such cases, the plurality of peaks may be identified from the displayed Raman spectrum, i.e. visually.

In some cases, the Raman spectral data may include at least one band-limited region of interest. A band-limited region of interest may be a region where peaks characteristic of MSU depositions are expected to be present. For example, a first band-limited region of interest may include a first region of interest from 500 cm⁻¹ to 700 cm⁻¹ and a second band-limited region may include a region of interest from 1000 cm⁻¹ to 1502 cm⁻¹. These regions of interest were determined, in accordance with the teachings herein, to represent regions where peaks associated with MSU may be expected with high intensity compared to any background noise.

The Raman spectral data can be filtered to exclude data outside of the at least one of the band-limited regions of interest. The plurality of peaks can then be identified in the Raman spectral data from the band-limited regions of interest. In cases where the peaks are identified visually/manually by a user of the device 10, displaying only the band-limited regions of interest may facilitate easier and more rapid identification of peaks associated with monosodium urate by focusing attention on those areas of the Raman spectrum where relevant peaks are expected.

At 250, if the number of identified peaks associated with monosodium urate is greater than a detection threshold, it may be determined that at least one monosodium urate deposition is present in the anatomical region. While the presence of MSU depositions will typically generate a large number of peaks associated with MSU, not all peaks may be present in Raman spectral data captured from the anatomical region because overlap or interference with Raman spectral data generated by other biological materials such as skin, fat, and cartilage for example. In some cases the detection threshold may be set to six peaks, such that when six peaks associated with monosodium urate are identified it will be determined that at least one monosodium urate deposition is present in the anatomical region that is being examined or evaluated.

In some embodiments, monosodium urate depositions may be determined to be present in the anatomical region when the identified plurality of peaks includes at least one of peaks at 1502 cm⁻¹, 1445 cm⁻¹, 1420 cm⁻¹, 1205 cm⁻¹, 1060 cm⁻¹, and 1010 cm⁻¹. In other embodiments, monosodium urate depositions may be determined to be present in the anatomical region when the identified plurality of peaks includes each of peaks at 1502 cm⁻¹, 1445 cm⁻¹, 1420 cm⁻¹, 1205 cm⁻¹, 1060 cm⁻¹, and 1010 cm⁻¹.

Reference will now be made to FIGS. 3A and 3B. FIG. 3A shows an example UA plot 300 illustrating the Raman spectrum of a uric acid standard, while FIG. 3B shows an example MSU plot 350 illustrating the Raman spectrum of lab grade MSU.

The largest peak shown in UA plot 300 was the peak at 1039 cm⁻¹. The largest peak shown in the MSU plot 350 is at 632 cm⁻¹, which closely mirrors the peak at 627 cm⁻¹ in the UA plot 300. In accordance with the teachings herein, it has been found that the UA plot 300 and the MSU plot 350 indicate the ability of Raman spectroscopy to differentiate uric acid and monosodium urate, while at the same time indicating that certain peaks may be similar between the Raman plots (e.g. the peak at 632 cm⁻¹ in the MSU plot 350 and the peak at 627 cm⁻¹ in the UA plot 300) using non-invasively obtained Raman data.

### Experimental Results

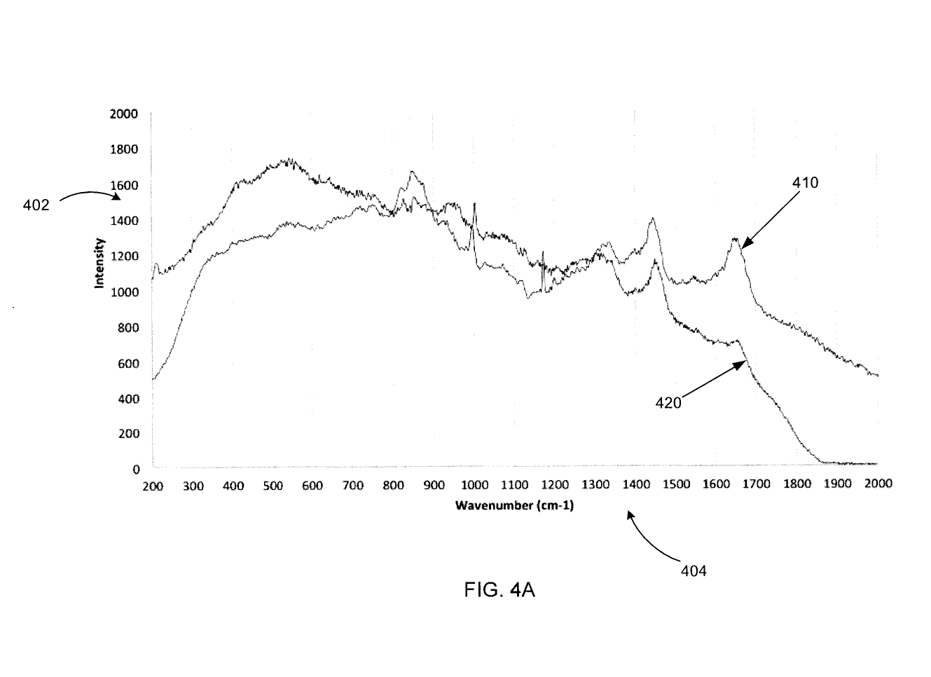

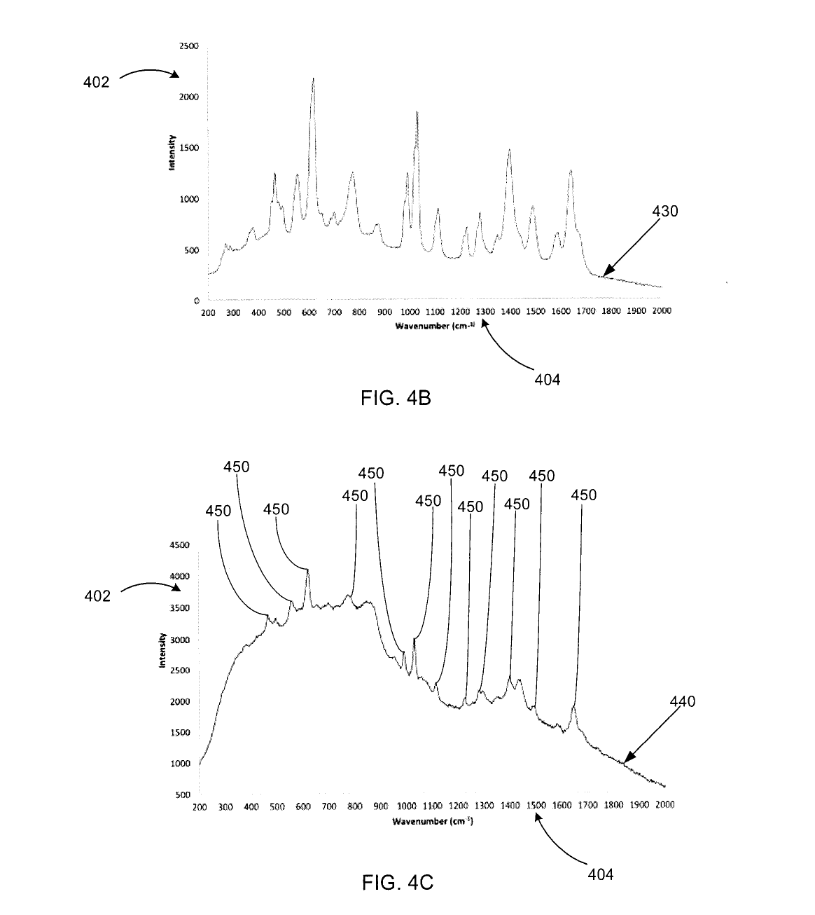

Referring now to FIGS. 4A-4C, shown therein are plots of Raman traces collected during preliminary tests conducted on an artificial knuckle of ayes (chicken) flesh (˜2 mm thickness) laid over UA salts. The traces shown in FIGS. 4A-4C indicate the different Raman spectrums generated for ayes flesh, bone through ayes flesh, UA as well as UA through ayes flesh. These traces indicate that Raman spectroscopy may be useful to identify the presence of sub-cutaneous uric acid. Given the similar peaks between UA and MSU, these tests indicated that MSU may be detectable subcutaneously using quick, relatively painless, non-invasive measurements.

A Raman spectrometer using a 785 nm wavelength laser with 100 mW power was used in these tests. Individual scans were conducted for ayes flesh, bone through ayes flesh, UA and UA through ayes flesh. FIGS. 4A-4C illustrate the Raman spectrums generated by each of these individual scans with a normalized Raman intensity 402 plotted against wavenumbers 404.

A piece of ayes flesh was cut in a rectangular shape with a thickness of ˜2 mm, enough to fill a Raman spectrometer holder. The ayes flesh was arranged such that the Raman laser would focus on the outer side of the flesh. The laser was then focused on the surface of the flesh and the characteristic Raman trace of the flesh was recorded with a scan time of 5 s. This was repeated 5 times and the average was obtained). Ayes flesh trace plot 410 shown in FIG. 4A illustrates an example of the characteristic Raman trace determined for the ayes flesh.

Subsequently, a second piece of ayes flesh containing bone and cartilage was cut. The bone lay just below the surface of the flesh and was visible using the naked eye. The Raman laser was focused on the bone through the flesh to obtain a Raman trace of the bone through ayes flesh with a 5 s scan time. This was repeated 5 times and the average was obtained. The bone through ayes flesh trace plot 420 shown in FIG. 4A illustrates an example of the Raman trace of the bone collected through ayes flesh.

Ayes flesh trace plot 410 and bone through ayes flesh trace plot 420 both show sharp peaks at ˜1000 cm⁻¹ and bone through ayes flesh trace plot 420 shows a sharp peak at ˜1180 cm⁻¹ all due to fluorescent light interference. These peaks are the result of interference with lower intensity traces such as the Ayes flesh trace plot 410 and bone through ayes flesh trace plot 420. These peaks do not appear in the Uric acid through chicken flesh trace plot 440 shown in FIG. 4C as the intensity of that trace is much greater.

A UA Raman trace was obtained by a covering the cleaned base of a Raman spectrometer holder with UA salts and scanning for 5 s. This was repeated 5 times and the average was obtained to obtain the Raman trace. The UA trace plot 430 shown in FIG. 4B illustrates an example of the characteristic Raman trace of the UA salts.

The original boneless cut of ayes flesh was then laid over the bed of UA and re-examined using Raman spectroscopy to detect the UA through the flesh. This was done by focusing the Raman laser below the surface of the flesh to detect the UA. Once the correct focus length through the flesh was obtained, a scan time of 5 s was used to obtain a good signal of the UA. This was repeated 5 times and the average was obtained. Uric acid through chicken flesh trace plot 440 shown in FIG. 4C illustrates the Raman spectrum that was acquired of the UA through the chicken flesh.

Uric acid through chicken flesh trace plot 440 illustrates that the UA peaks 450 are identifiable underneath the ˜2 mm of chicken flesh, which is considerably thicker than the flesh present on a patient's phalange joints. These preliminary results indicated that embodiments of Raman laser spectroscopy techniques applied in accordance with the teachings herein can be used to identify UA crystals below the surface of skin through flesh in humans and animals.

As shown above in FIGS. 3A and 3B, the Raman trace of UA differs slightly to that of MSU, which is the main mineral that is expected to indicate the presence of gout in the joints. However, UA has similar Raman peaks to MSU (for example, see FIGS. 3A and 3B).

The largest peak detected in the uric acid trace 410 in FIG. 4 was the 632 cm⁻¹ peak and not the 1039 cm⁻¹ peak which had the greatest intensity in plot 300. The 632 cm⁻¹ peak is also mirrored with a similar intensity at 627 cm⁻¹ in the MSU scan shown in plot 350. This suggests that detection of MSU using this peak may also be achieved through 1.5 mm of ayes flesh due to this mirrored peak. Other peaks were subsequently identified through further trials that can also be used to identify MSU depositions similar to the numerous UA peaks 450 identified in the UA trace 410 (see, for example, FIGS. 5 and 6, discussed below).

Subsequently, the inventors created an artificial knuckle consisting of porcine flesh (>1.5 mm thick). A clinically sourced sample of tophus milk was deposited underneath the knuckle (being approximated by porcine flesh, which is thicker than human flesh) by syringe. Raman Spectroscopy was performed on the other side of the knuckle from the milk using the Sierra Reader spectroscopy device mentioned above. In accordance with the teachings herein, it has been determined that peaks associated with MSU are detectable through an artificial knuckle.

Referring now to FIG. 5, shown therein is an example plot 500 illustrating the Raman intensity 510 for a range of wavenumbers 520 from a Raman spectroscopic scan of aspirated MSU containing fluid from the elbow of a patient diagnosed with gout. The Sierra Reader™, mentioned above, was used to analyze samples of aspirated tophi milk secured from a rheumatology clinic.

Tophi are nodular masses of MSU crystals deposited in soft tissues of the body. They are a late complication of hyperuricemia and develop in more than half of patients with untreated gout. Tophus milk is an aspirated fluid that contains a very high build-up of MSU crystals. Plot 500 in FIG. 5 shows a typical Raman spectrum that resulted from analysis of the tophus milk samples from the rheumatology clinic. The peaks 530 correspond to the presence of MSU crystals.

An initial trial with clinically diagnosed gout sufferers was also conducted to determine if the Raman spectroscopy systems and methods described herein may be used to non-invasively identify the presence of sub-cutaneous MSU crystal depositions in humans. Referring now to FIG. 6, shown therein is an example plot 600 illustrating the Raman intensity 610 for a range of wavenumbers 620 from a Raman spectroscopic scan of the metatarsophalangeal joint of patient clinically diagnosed with gout. The plot 600 illustrates the resulting Raman spectral data unfiltered, including contributions from the patient's skin, fat cells and blood. As can be seen from plot 600, the peaks 630 and 635 are slightly shifted with respect to the peaks seen in lab grade MSU, as shown above in FIG. 3B.

The trial resulted in 100% sensitivity and specificity in determining the presence of MSU crystals in the gout sufferers, compared to controls. The peaks 630 and 635 were both found to be indicative of MSU deposition. The peaks 635 (listed in table 1 below) were visible in all patients tested and absent in all control subjects.

TABLE 1
MSU Peaks
Wavenumber (cm⁻¹)
1502
1445
1420
1205
1060
1010

Table 1 lists bands that were identified in the Raman spectral data obtained from all clinically diagnosed gout sufferers and absent in all controls. At least one of peaks 635, peaks 630 or a combination of a threshold detection number of MSU peaks (630 or 635) can be used as markers for the presence of MSU crystal deposition, and thus identifiers for the presence of gout or other MSU-related conditions. As mentioned above, a range around each of these peaks (630 or 635) can be used to account for slight shifts in the Raman spectral data that may occur in some patients. For example, a range of +/−5 cm⁻¹ may be used.

In some cases, a detection threshold may be selected by a user to ensure sufficient confidence in the determination of MSU depositions. For example, in some embodiments the detection threshold may be set as at least one of the peaks 635 listed in table 1. In other embodiments, the detection threshold may be set as all of the peaks 635 listed in table 1. Other peaks associated with MSU, such as peaks 630 can also be used to determine that gout is present in an anatomical region. Accordingly, the detection threshold can also be set as a minimum number of peaks 630, or as a minimum number of peaks 630 and peaks 635 associated with MSU.

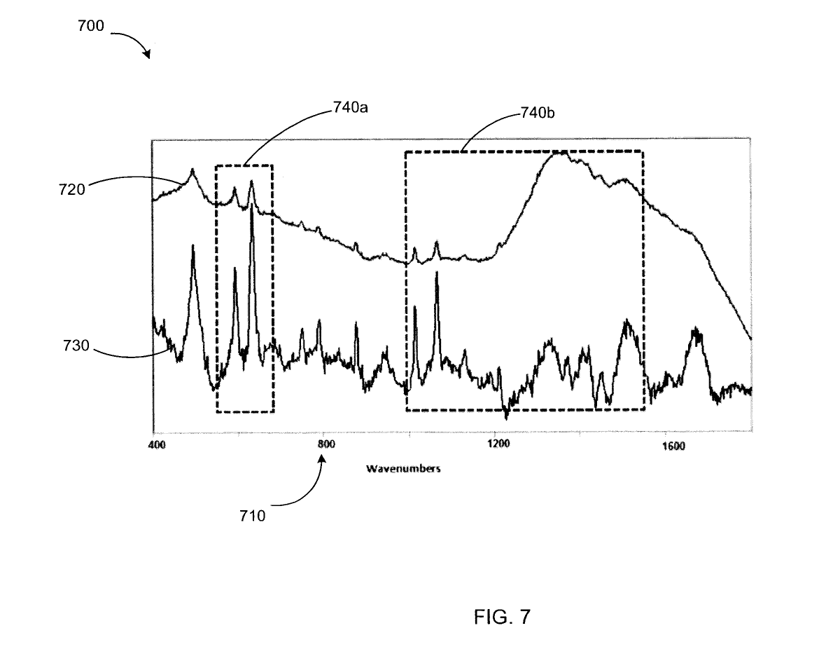

Referring now to FIG. 7, shown therein is an example plot 700 showing the form of unfiltered Raman spectral data 720 and filtered Raman spectral data 730. The unfiltered Raman spectral data 720 demonstrates the interference from water (1200 to 1800 cm⁻¹) and minor protein peaks not associated with MSU. The filtered Raman spectral data 730 was generated from the unfiltered Raman spectral data 720 using various commercially available filtering software suites used with Raman spectroscopy devices, such as those included with the Sierra Series™ range of Raman spectrometers developed by Snowy Range Instruments® mentioned above.

The plot 700 also illustrates a first band limited region of interest 740a and a second band limited region of interest 740b. In some cases, the Raman spectral data includes at least one of these band-limited regions of interest and the plurality of peaks associated with monosodium urate will be identified in the at least one band-limited region of interest. The regions may be selected as those regions that tend to contain the most intense peaks of MSU. In the example shown in plot 700, the first band limited region of interest 740a ranges from 500 cm⁻¹ to 700 cm⁻¹ and while the second region of interest ranges from 1000 cm⁻¹ to 1502 cm⁻¹.

The regions of interest may be identified and used to simplify the process of identifying peaks associated with gout. The Raman spectral data may be filtered to exclude data from outside the regions of interest. The peaks associated with MSU can then be identified from the Raman spectral data in the regions of interest. In some cases, only those portions of the Raman spectrum determined for an anatomical region in the regions of interest may be displayed to a user. This may allow the user to more rapidly and easily identify peaks associated with MSU and gout or other MSU-related conditions.

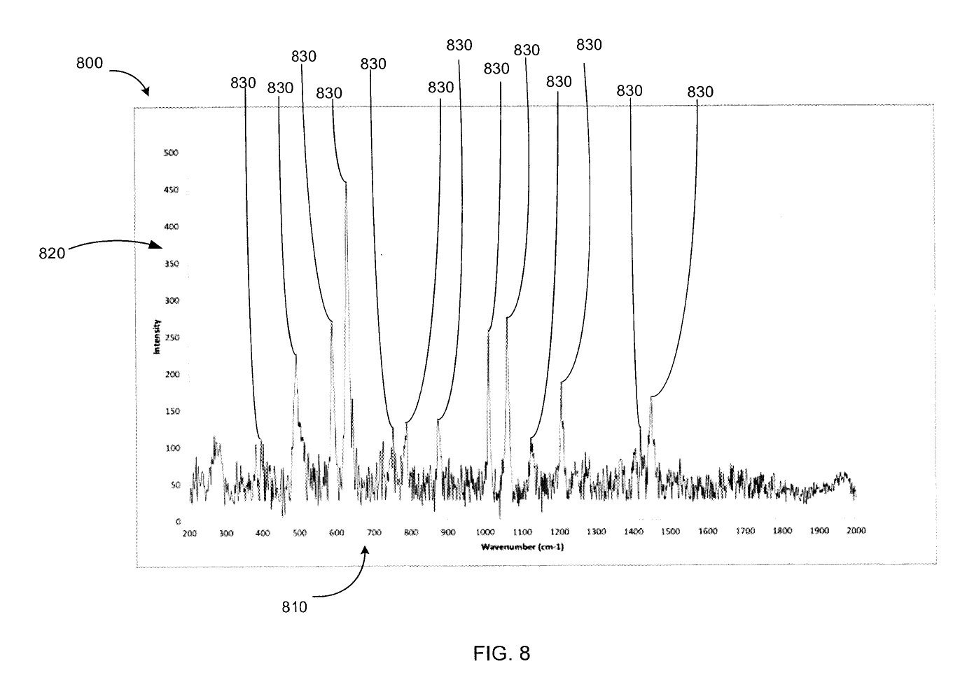

Referring now to FIG. 8, shown therein is an example plot 800 illustrating a Raman spectrum determined based on Raman spectral data obtained from tophi of a patient suffering from gout. The Raman spectrum shown in plot 800 was obtained in accordance with the methods described herein, in particular acts 210-230 of method 200 described above. Plot 800 illustrates the Raman intensity 810 for a range of wavenumbers 820 included in the collected Raman spectral data.

As mentioned above, tophi are nodular masses of MSU crystals deposited in soft tissues of the body. A plurality of peaks 830 associated with MSU can be identified in the plot 800. As mentioned above the peaks 830 show a slight shift with respect to the Raman spectrum of the lab grade MSU shown above in FIG. 3B. However, the presence of an MSU deposit can nonetheless be determined based on the plot 800 in accordance with the teachings herein.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without generally departing from the embodiments described herein.

1. A method of detecting monosodium urate depositions in an anatomical region, the method comprising:
irradiating a portion of the anatomical region through a skin surface using a light source;
receiving scattered light from the portion of the anatomical region;
determining Raman spectral data from the received scattered light;
identifying a plurality of peaks associated with monosodium urate in the Raman spectral data; and
determining that a monosodium urate deposition is present in the anatomical region when the number of identified peaks associated with monosodium urate is greater than a detection threshold.

2. The method as defined in claim 1, wherein the plurality of peaks associated with monosodium urate comprise at least one of peaks at 1502 cm⁻¹, 1445 cm⁻¹, 1420 cm⁻¹, 1205 cm⁻¹, 1060 cm⁻¹, and 1010 cm⁻¹.

3. The method as defined in claim 1, wherein the detection threshold is at least 6 peaks.

4. The method as defined in claim 3, wherein the monosodium urate deposition is determined to be present in the anatomical region when the identified peaks include each of the peaks at 1502 cm⁻¹, 1445 cm⁻¹, 1420 cm⁻¹, 1205 cm⁻¹, 1060 cm⁻¹, and 1010 cm⁻¹.

5. The method as defined in claim 1, wherein the acts of irradiating, receiving scattered light, and determining Raman spectral data are performed at least twice, and the method further comprises averaging the determined Raman spectral data; and wherein the plurality of peaks associated with monosodium urate are identified from the averaged Raman spectral data.

6. The method as defined in claim 1, further comprising displaying a Raman spectrum based on the determined Raman spectral data, wherein the plurality of peaks associated with monosodium urate are identified from the displayed Raman spectrum.

7. The method as defined in claim 1, wherein:
the Raman spectral data is filtered to exclude data outside of at least one band-limited region of interest; and
the plurality of peaks associated with monosodium urate are identified in the Raman spectral data from the filtered at least one band-limited region of interest.

8. The method as defined in claim 7, wherein the at least one band-limited region of interest comprises at least one of a first region of interest from 500 cm⁻¹ to 700 cm⁻¹ and a second region of interest from 1000 cm⁻¹ to 1502 cm⁻¹.

9. A method of detecting monosodium urate depositions in an anatomical region, the method comprising:
irradiating the anatomical region using a light source;
receiving scattered light from the anatomical region;
determining Raman spectral data associated with the received light, wherein the Raman spectral data includes at least one of a first region of interest from 500 cm⁻¹ to 700 cm⁻¹ and a second region of interest from 1000 cm⁻¹ to 1502 cm⁻¹;
identifying a plurality of peaks in the at least one of the first region of interest and the second region of interest; and
determining that a monosodium urate deposition is present in the anatomical region when the identified plurality of peaks includes at least one peak associated with monosodium urate.

10. The method as defined in claim 9, wherein the Raman spectral data includes the first region of interest from 500 cm⁻¹ to 700 cm⁻¹ and the second region of interest from 1000 cm⁻¹ to 1502 cm⁻¹.

11. The method as defined in claim 9, wherein the monosodium urate deposition is determined to be present when the identified plurality of peaks includes at least one of peaks at 1502 cm⁻¹, 1445 cm⁻¹, 1420 cm⁻¹, 1205 cm⁻¹, 1060 cm⁻¹, and 1010 cm⁻¹.

12. The method as defined in claim 9, wherein the monosodium urate deposition is determined to be present when the identified plurality of peaks includes each of peaks at 1502 cm⁻¹, 1445 cm⁻¹, 1420 cm⁻¹, 1205 cm⁻¹, 1060 cm⁻¹, and 1010 cm⁻¹.

13. The method as defined in claim 9, wherein the acts of irradiating, receiving light, and determining Raman spectral data are performed at least twice, and the method further comprises averaging the determined Raman spectral data, wherein the plurality of peaks are identified from the averaged Raman spectral data.

14. The method as defined in claim 9, wherein:
the Raman spectral data is filtered to exclude data outside of the at least one of the first region of interest and the second region of interest; and
the plurality of peaks are identified from the filtered Raman spectral data.

15. The method as defined in claim 9, further comprising displaying a Raman spectrum based on the determined Raman spectral data, wherein the plurality of peaks are identified from the displayed Raman spectrum.

16. A hand-held device for detecting monosodium urate depositions in an anatomical region of a subject, wherein the device comprises:
a light source configured to irradiate the anatomical region through a skin surface of the subject;
a light collection device configured to receive scattered light from the anatomical region;
a spectral selection device coupled to the optical detection component and configured to separate and select Raman scattered light from the scattered light collected by the light collection device; and
a data analysis module for determining Raman spectral data from the Raman scattered light and identifying at least one peak of interest in Raman spectral data associated with monosodium urate.

17. The device of claim 16, wherein the data analysis module is configured to determine the monosodium urate depositions when the at least one peak of interest comprises at least one peak at 1502 cm⁻¹, 1445 cm⁻¹, 1420 cm⁻¹, 1205 cm⁻¹, 1060 cm⁻¹, and 1010 cm⁻¹.

18. The device of claim 16, wherein the data analysis module is configured to determine that a monosodium urate deposition is present in the anatomical region when the number of identified peaks associated with monosodium urate is greater than a detection threshold, wherein the detection threshold is at least 6 peaks.

19. The device of claim 16, wherein the light source is adjusted to have a focus at a subcutaneous portion of the anatomical region of interest comprising at least one of a joint including a toe, a finger, an elbow or a knee; cartilage; an ear and an eyebrow.

20. The method of claim 1, wherein the portion of the anatomical region that is irradiated comprises at least one of a joint including a toe, a finger, an elbow or a knee; cartilage; an ear and an eyebrow.
